(19) United States
(12) Patent Application Publication
(10) Pub. No.: US 2015/0119986 A1
(43) Pub. Date: Apr. 30, 2015

(54) SYSTEM AND METHOD FOR PERFORMING MINIMALLY INVASIVE FACELIFTS

(71) Applicant: Francisco PICO-Fazzi, Plymouth Meeting, PA (US)

(72) Inventor: Francisco PICO-Fazzi, Plymouth Meeting, PA (US)

(21) Appl. No.: 14/523,438

(22) Filed: Oct. 24, 2014

Related U.S. Application Data
(60) Provisional application No. 61/895,214, filed on Oct. 24, 2013.

(51) Int. Cl. A61F 2/00

(52) U.S. Cl. CPC A61F 2/0059
USPC 623/15.12

(57) ABSTRACT

A system for performing minimally invasive facelifts is provided. The system includes a plate having a base having a top surface and an opposing bottom surface. The base has a plurality of through holes formed therein that extends between the top surface and the bottom surface. A plurality of barbs extends outwardly from the bottom surface. At least one anchor screw is adapted to secure the plate to the bone of patients. A method for performing minimally invasive facelifts is also provided.

### CROSS-REFERENCE TO RELATED APPLICATION

This application claims priority from U.S. Provisional Patent Application Ser. No. 61/895,214, filed on Oct. 24, 2013, the contents of which are incorporated herein by reference in its entirety.

### FIELD OF THE INVENTION

The present invention relates to a system and method for performing minimally invasive facelifts and other types of cosmetic surgery.

### BACKGROUND OF THE INVENTION

Presently, as shown in the sectional view of bone and soft tissue, representing a portion of a patient's face, of FIG. 1, facelift procedures are presently performed by making an incision or puncture through a patient's skin 18 from the exterior and inserting a lifting device (shown by arrow “A”), or dissecting, resecting and excising tissue after dissecting through layers of fat and the superficial muscular aponeurotic system (“SMAS”) 20, which is located between layers of fat 22 and muscle 24. This procedure poses several potential problems, conventional facelifts are very invasive and require excision of vital tissues, including the possibility of the lifting device being inserted too deep into the SMAS 20 and engaging, and subsequently damaging muscle 24, nerves, or skin if too superficial, as well as potential scarring from the incision, or being too superficial and thus perforating skin or patient able to palpate or notice the implant through the skin.

It would be beneficial to provide a system and a method for performing a minimally invasive facelift that eliminates the potential for visible scarring at the incision site, as well as reducing the likelihood of inadvertently engaging and possibly damaging muscle or nerves.

### BRIEF SUMMARY OF THE PRESENT INVENTION

Briefly, the present invention provides a system for performing minimally invasive facelifts. The system comprises a plate having a base having a top surface and an opposing bottom surface. The base has a plurality of through holes formed therein that extends between the top surface and the bottom surface. A plurality of barbs extends outwardly from the bottom surface. At least one anchor screw is adapted to secure the plate to the bone of patients.

Additionally, the present invention provides a hook for performing facelifts. The hook comprises a generally cylindrical body extending along a longitudinal axis. The body has a first end having a tip extending away from the body along the longitudinal axis and a second end having a through opening formed therein. A plurality of barbs extends from the body. A suture having a first end extends through the through opening and secured to the second end.

Further, the present invention provides a hook for performing facelifts comprising an elongate, generally planar body extending along a longitudinal axis. The body has a first face and a second face, opposing the first face. The body has a plurality of through openings extending between the first face and a second face. A first plurality of barbs extends outwardly from the first face in a first direction generally along the longitudinal axis. A suture is fixedly connected to an end of the body.

The present invention further provides a method performing a non-invasive facelift procedure on a patient. The method comprises the steps of making an incision inside the patient's mouth in intraoral mucosa, above bone; inserting a plate into the incision and securing the plate to the bone; inserting hook percutaneously through exterior of the patient's skin, through fat, SMA, muscle, and fat slap deep fat pad; and manipulating the hook to a desired location, and, pulling the hook backward to lift the skin a desired amount.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a sectional view of bone and soft tissue showing the location of the procedure for performing facelifts according to the prior art;

FIG. 2 is a perspective view of a universal mounting plate used to perform facelifts according to an exemplary embodiment of the present invention;

FIG. 3 is a perspective view showing the plate of FIG. 2 mounted on the cheekbone of a patient in accordance with the present invention;

FIG. 4 is a perspective view of a malar/submalar/zygomatic mounting plate used to perform facelifts according to an exemplary embodiment of the present invention;

FIG. 4A is a first exemplary facial implant reservoir used with or without the plate shown in FIG. 4;

FIG. 4B is a second exemplary facial implant reservoir used with or without the plate shown in FIG. 4;

FIG. 4C is a third exemplary facial implant reservoir used with or without the plate shown in FIG. 4;

FIG. 5 is a perspective view showing the plate of FIG. 4 mounted on the cheekbone and jawbone of a patient in accordance with the present invention;

FIG. 6A is a first exemplary embodiment of a mental/parasymphaseal mounting plate used to perform facelifts according to an exemplary embodiment of the present invention;

FIG. 6B is a second exemplary embodiment of a mental/parasymphaseal mounting plate used to perform facelifts according to an exemplary embodiment of the present invention;

FIG. 6C is a third exemplary embodiment of a mental/parasymphaseal mounting plate used to perform facelifts according to an exemplary embodiment of the present invention;

FIG. 7 is a perspective view showing the plate of FIG. 6C mounted on the chin of a patient in accordance with the present invention;

FIG. 8 the perspective view of a first exemplary embodiment of a lifting insert to be used in a SMAS or below muscle in a patient according to the present invention;

FIG. 9 is a perspective view of a second exemplary embodiment of a lifting insert according to the present invention;

FIG. 9A is an enlarged view of the barbed ribbon of the transcutaneous lifting insert shown in FIG. 9;

FIG. 10 is a perspective view of a ribbon lifting insert according to the present invention;

FIG. 10A is an enlarged view of a portion of the ribbon lifting insert shown FIG. 10;

FIG. 11 is a perspective view of a cannula assembly used to insert either of the lifting inserts shown in FIGS. 8 and 9;

FIG. 11A is a perspective view of a cannula insert with a barbed end used to aid in a repositioning process according to an exemplary embodiment of the present invention;

FIG. 12 is a perspective view of an insertion assembly used to insert the lifting ribbon insert shown in FIG. 10;

FIG. 12A is a perspective view of a cannula insert with a barbed end that can be inserted through the insert assembly shown in FIG. 12; and

FIG. 13 is a sectional view of bone and soft tissue showing the location procedure for performing facelifts according to the present invention.

### DETAILED DESCRIPTION OF THE INVENTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

The present invention includes a system and a method for performing minimally invasive and non-invasive facelifts on a patient. Most, if not all, insertions are performed via an incision through the inside of patient's mouth so that minimal or no external skin or tissue scarring takes place. Typical lift procedures according to the present invention are performed on the mid-face of a patient, which can be defined as the area of the face from the corner of the eyes and below. By way of example, such as facelift may include implant on the cheekbones to better define the patient's cheeks and lifts below the eyes to eliminate wrinkles.

The inventive system includes several embodiments of at least one plate that is affixed to bone structure, such as a skull of the patient, to a first end of which at least one suture can be attached. A second end is attached to a barb that engages and lifts soft tissue to perform the lift procedure. The lift procedure can be performed mid-face, neck, or brow.

The inventive procedure is a nonexcisional, minimally invasive repositioning of facial tissue to a more natural state in order to compensate for deflation and descent as a result of aging. Anchoring devices are implanted that can be placed on bone intraorally and guided via cone beam scan CT and small cannular percutaneous insertion.

This intraoral approach eliminates the need for hydrodissection and percutaneous infiltration of local anesthesia, thus providing a more accurate and symmetrical evaluation and as a result, less swelling, pain, and the possibility of being too deep or too shallow, thereby blindly damaging tissue. The use of cone beam CT guidance ensures proper placement and visualization of anatomical structures. The inventive procedure is reversible and can also be repeated over time. The inventive anchoring implant can also be used in combination with the anchoring percutaneous “lifting lines” or by itself, depending on the goals and situation of the lift.

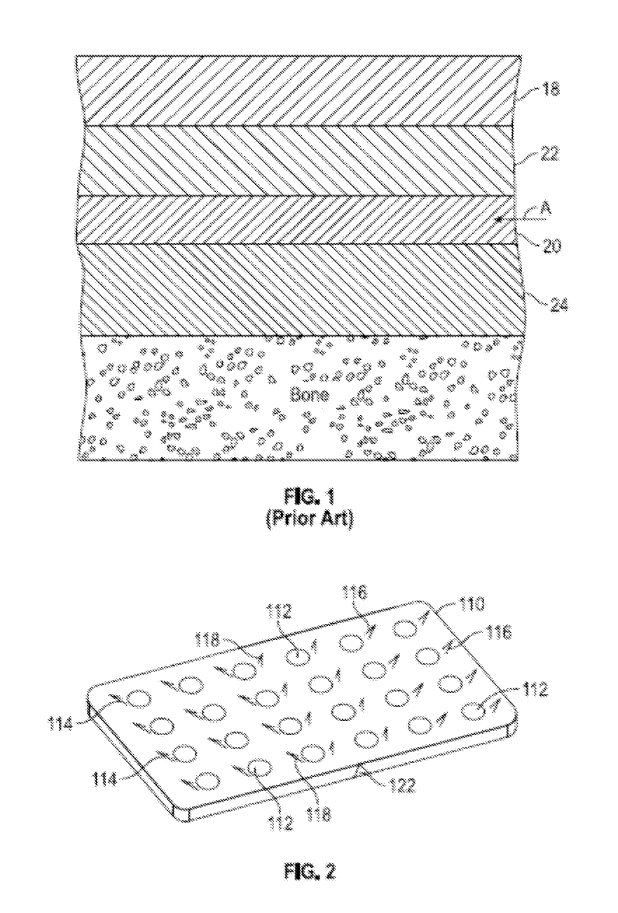

Referring to FIGS. 2-12, the inventive system and a method of using the inventive system to perform a minimally invasive facelift procedure are shown. Referring specifically to FIG. 2, the inventive system includes a universal, generally flat, planar base plate 110 that can be cut, shaped, and molded, and thereby customized for a particular patient, to conform to a bone structure to which plate 110 is to be attached. Plate 110 has a thickness of not greater than 1.5 mm and can be manufactured from a resorbable material such as, for example, polylactic acid. Alternatively, plate 110 can be constructed from a non-resorbable material, such as, for example, polyvinyl, polypropylene, titanium, or stainless steel.

Plate 110 includes a plurality of through holes 112 extending between a top surface 121 and a bottom surface 122 that provide for anchoring plate 110 to a bone via screws (not shown) or other known connecting devices. Through holes 112 also provide anchoring for a facelift suture. Through holes 112 may be arranged in a grid pattern, as shown in FIG. 2. Alternatively, through holes 112 may be arranged in an apparent random pattern.

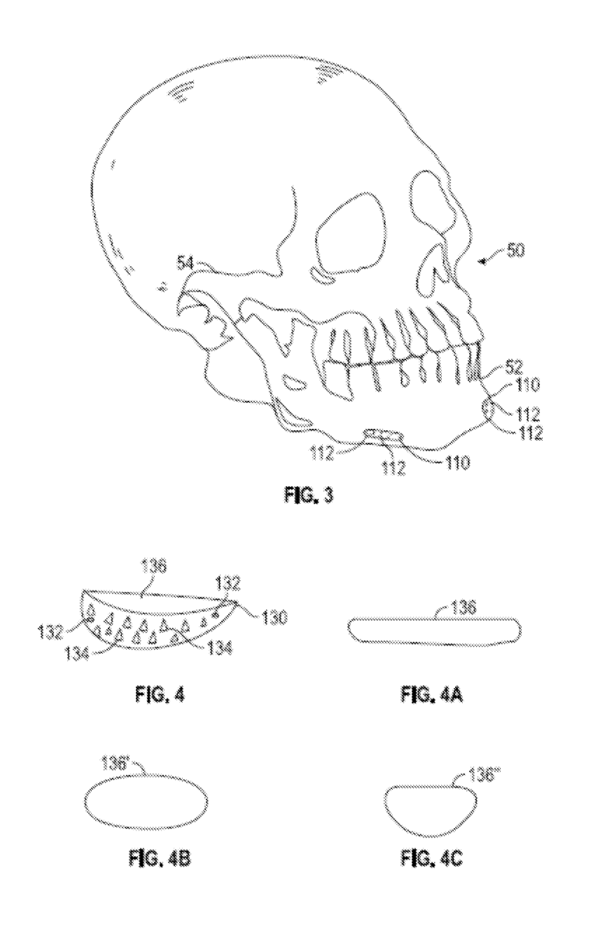

Plate 110 also includes a plurality of barbs 114-118 that extend outward from a bottom surface 122 of plate 110. Barbs 114-118 may extend in different directions from bottom surface 122. For example, as shown FIG. 3, barbs 114 may be angled at about 45 degrees upward from plate 110 in a first direction. Barbs 116 may be angled about 45 degrees upward from plate 110 in a second direction, away from the first direction. Barbs 118 may extend in a third direction, extending generally orthogonally away from plate 110. The different directions of barbs 116-118 assist in anchoring plate 110 to a bone structure, such as, a lower jaw 52 or upper jaw 54 of the patient 50, as shown in FIG. 3.

Depending on the size, location, and physical contours of the bone structure to which plate 110 is to be attached, the surgeon can cut plate 110 in a desired size and shape to obtain such contours, desired locations of through holes 112, and directions of barbs 116-118 to ensure proper anchoring of plate 110 into the bone structure of the patient 50. Plate 110 can be anchored into the bone with a known removable adhesive means, such as, for example, anchor screws (not shown). The use of an adhesive means, such as anchor screws, allows plate 110 to be removed and repositioned at a later date, if desired.

The inventive system also includes a malar/submalar/zygomatic plate 130, shown in FIG. 4, that can be used to enhance a cheek bone 54 of patient 50, as shown FIG. 5. While plate 110 is generally planar and extends in generally only two dimensions, plate 130 is three dimensional. As used herein, the term “three-dimensional” is used to describe a surface that extends in three orthogonal planes. Plate 130 uses silicone, saline, hyluronic acid, or other suitable implant material applied thereto, to provide “depth” to plate 130 and, as a result, to cheek bone 54.

Referring back to FIG. 4, plate 130 includes a plurality of through holes 132 and barbs 134, similar to through holes 112 and barbs 116 in plate 110, as described above. Plate 130 also includes a filler 136 into which a desired amount of material is injected to provide a desired depth to plate 130. Filler implant 136 may be provided in at least one of several shapes, including, but not limited to, tubular, oval and triangular, as shown in exemplary filler implant reservoirs 136, 136′, and 136″, shown in FIGS. 4A-4C, respectively.

Plate 130 can be anchored into the bone with a known removable adhesive means, such as, for example, anchor screws (not shown). The use of an adhesive means, such as anchor screws, sutures, etc., allows plate 130 to be removed and repositioned at a later date, if desired. An exemplary location of installation of plate 130 in cheek bone 54 is shown FIG. 5. In this example, plate 130 incorporates triangular filler implant 136″.

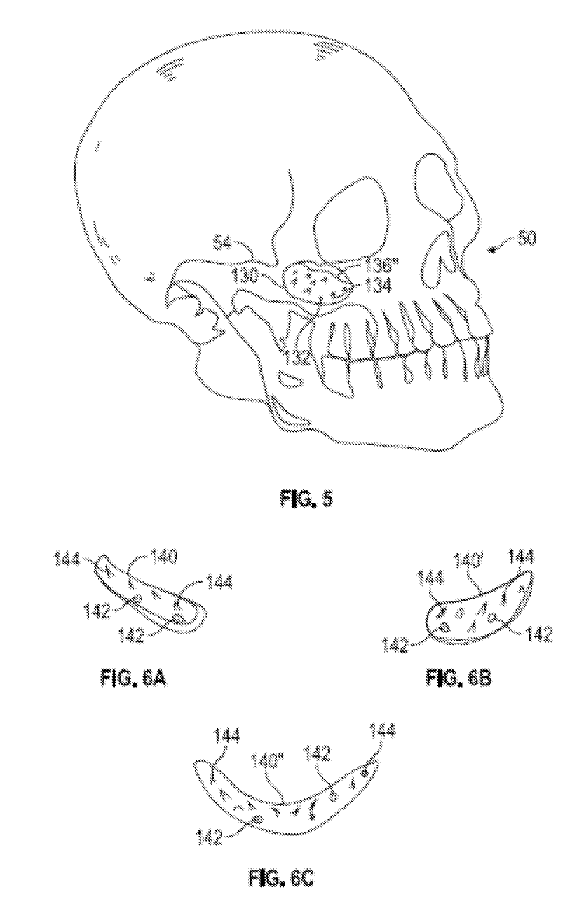

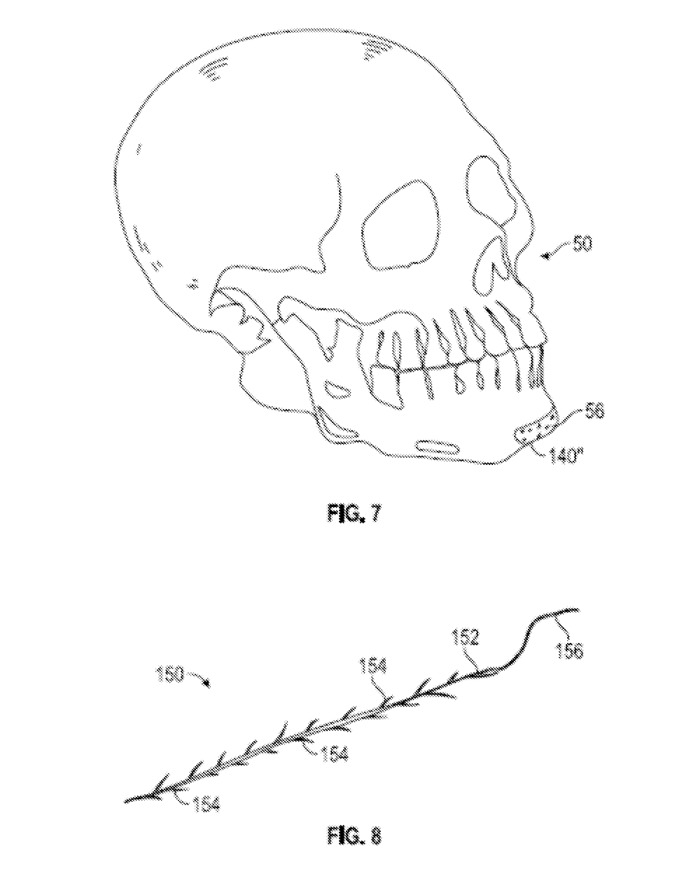

The inventive system also includes symphaseal/parasymphaseal plates 140, 140′, 140″, shown in FIGS. 6A, 6B, and 6C, respectively, that can be used to enhance a chin 56, as shown FIG. 7. Similar to plates 110 and 130, each of plates 140, 140′, 140″ includes a plurality of through holes 142 and barbs 144.

The inventive system further includes a plurality of lifting hooks 150, 160, 170 that can be used to engage and re-position soft tissue to perform a desired lift. Hooks 150, 160, 170 can be radiopaque or radiolucent and can also be manufactured from a resorbable or a non-resorbable material, similar to plate 110 described above.

Referring to FIG. 8, hook 150 includes an elongate, generally cylindrical hook body 152 extending along a longitudinal axis 153. Hook body 152 has a first end 155 having a tip extending away from body 152 along longitudinal axis 153 and a second end 157 having an opening extending therethrough. The tip can be a sharpened tip or a blunt tip. A plurality of barbs 154 that extend outward and rearward from hook body 152 toward second end 157. Barbs 154 are staggered around the outer periphery of hook body 152. Barbs 154 can be angled at about a 45 degree angle with respect to hook body 152. A suture 156 extends from a second end 157 of hook body 152. Suture 156 enables the surgeon to manipulate hook 150 to perform the desired amount of lift of the flesh being lifted. In the event that hook 150 is desired to be removed from patient 50 after insertion, hook 150 is advanced forward (in a direction away from suture 156) so that barbs 154 do not engage and tear into soft tissue. Hook 150 can be radiopaque or radiolucent, depending on the desires of the particular physician.

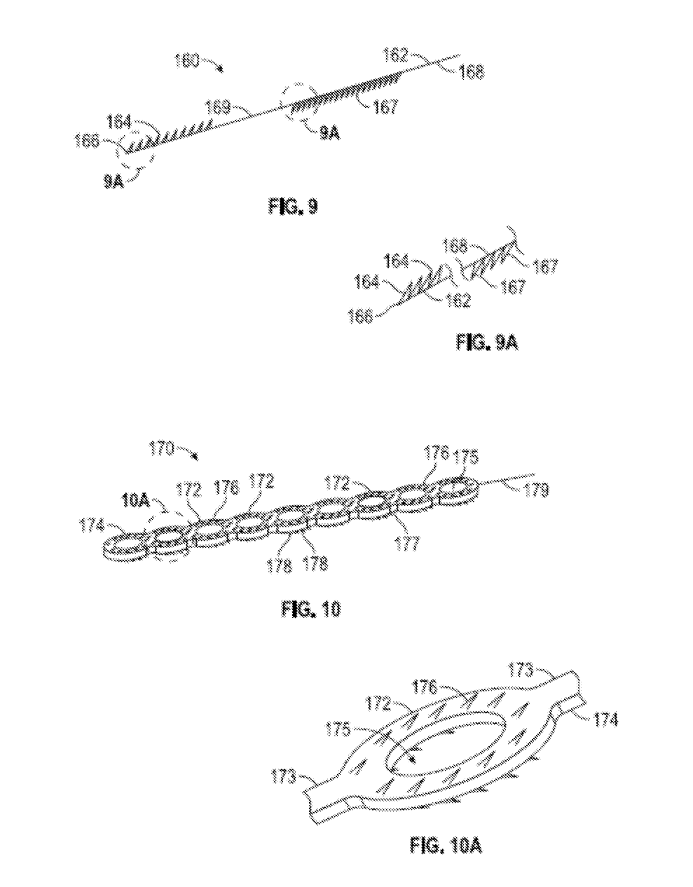

As shown in FIGS. 9 and 9A, hook 160 has a hook body 162 and a first plurality of barbs 164 that extend outward and rearward from a first end 166 of hook body 162. Hook 160 also has a second plurality of barbs 167 that extend outward and forward from a second end 168 of hook body 162. Barbs 164, 167 are staggered around the outer periphery of hook body 162. Hook body 162 includes a midportion 169 between first and 166 and second end 168 that is devoid of any barbs. In the event that hook 160 is desired to be removed from patient 50 after insertion, hook 160 can be cut at midportion 169 so that the first end 166 of hook body 162 and be advanced forward and second end 168 of hook body 162 can be advanced rearward so that none of barbs 164 or barbs 167 engage and tear into soft tissue.

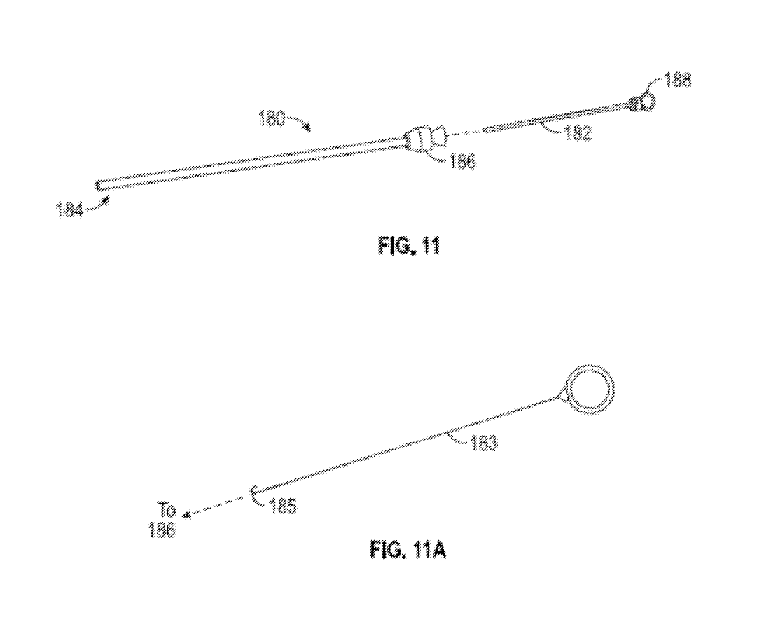

A ribbon hook 170 is shown in FIG. 10. Ribbon hook 170 is a generally elongate, planar structure extending along a longitudinal axis 171 and having a plurality of generally oval pads 172 that are attached to each other in series. As shown FIG. 10A, a connecting portion 173 can be used to connect each of adjacent pads 172. Each pad 172 includes an opening 175 extending therethrough along axis 171, which allows for a fixation device, such as a screw, a tack, a suture, or other anchoring device (not shown) to be used to secure ribbon hook 170 in place. In an exemplary embodiment, ribbon hook 170 is between approximately 0.25 mm thick and approximately 1.0 mm thick.

A first face 174 of ribbon hook 170 includes a first plurality of barbs 176 that extend outwardly from first face 174 at about a 45° angle in a first direction relative to longitudinal axis 171 of ribbon hook 170. A second face 177, disposed away from first face 174, includes a second plurality of barbs 178 that extend outwardly from second face 177 at about a 45° angle in a second direction, different from the first direction along longitudinal axis 171 relative to ribbon hook 170. A suture 179 is connected to one end of ribbon hook 172. Suture 179 allows the surgeon to manipulate ribbon hook 170 during the insertion and lifting process. As shown in FIG. 10, barbs 178 are present along only about half of the length of ribbon hook 170, beginning at the end of ribbon hook 170 that is connected to suture 179. According to the needs of the patient or the desires of the surgeon, ribbon hook 170 can be cut along a desired length before implantation. Further, in an alternative embodiment, not shown, ribbon hook 170 can be provided without reverse barbs 178.

Hooks 150, 160, 170 can be manufactured from a resorbable material, such as, for example, polylactic acid or collagen, such that hooks 150, 160, 170 eventually dissolve, so that hooks 150, 160, 170 do not have to be manually removed. The material from which hooks 150, 160, 170, is manufactured can be selected to provide a predetermined duration of time over which the hooks and last prior to being resorbed by the patient's body. Additionally, hooks 150, 160, 170 can be bendable and/or flexible in order to conform to the contours of the face of a particular patient.

Hooks 150 and 160 can each be introduced via an introducer cannula, such as, for example, an 18-30 gauge cannula 180, shown in FIG. 11. Cannula 180 can have varying lengths, such as, between about 1 cm and about 5 cm long. A cannula obturator 182 may be inserted into cannula 180 during insertion of cannula 180 in order to prevent a tissue from entering and clogging the cannula 180. Cannula obturator 182 is removed from cannula 180 prior to inserting hook 150, 160 into cannula 180 for insertion into the patient.

Prior to inserting cannula 180, a needle (not shown) having the same gauge size as cannula 180 is used to puncture the skin to the desired plane and removed. Then, cannula 180 is inserted into the puncture site. Cannula 180 includes a rounded blunt tip 184 at a first end of cannula 180. A female luer connection 186 can be located at a second end of cannula 180, distal from tip 184. Luer connection 186 is used to releasably couple to a male luer connection 188 on obturator 182 to keep obturator 182 within cannula 180 during insertion. Referring to FIGS. 11 and 11A, cannula 180 can be used with either of blunt obturator 182 or hooked obturator 183. Hooked obturator 183 having a hook 185, located at a distal end thereof, can be used to lift tissue and allow the physician to mark the skin prior to a final lift to determine the proper amount and vector of lift to perform. If satisfied, the physician advances obturator 183 forward, away from the incision, to disengage hook 185 from the tissue, then fishes cannula 180 forward to engage hook 185 within cannula 180 so hook 185 can be withdrawn through the cannula 180 without the hook 185 engaging tissue. The physician removes obturator 183 and inserts hook 150 or 160.

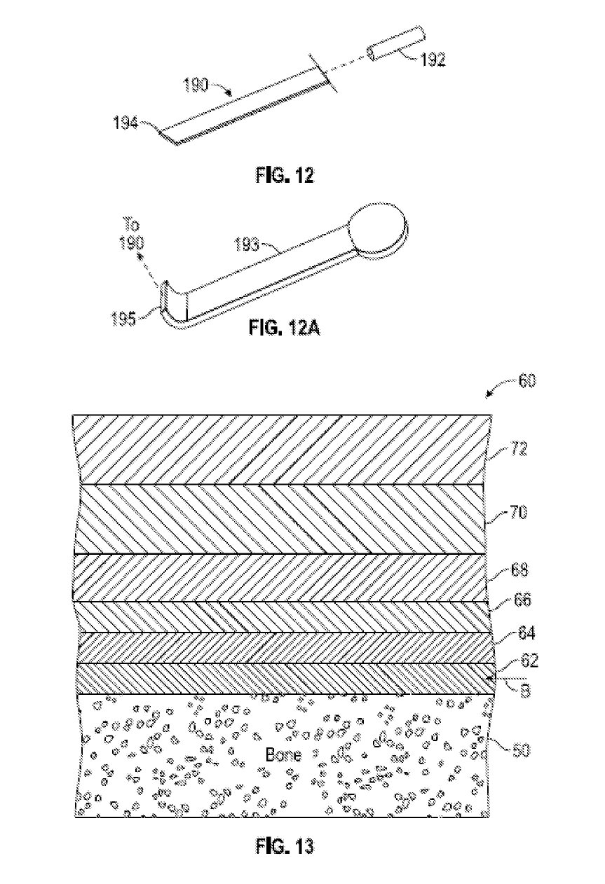

Hook 170 can be introduced via an introducer cannula 190, shown FIG. 12. Similar to cannula 180, cannula 190 can have varying lengths, such as, between about 1 cm and about 5 cm long. Introducer cannula 190 has a generally flat profile to accommodate the flat profile of ribbon hook 170. A cannula obturator 192 may be inserted into cannula 190 during insertion of cannula 190 in order to prevent a tissue from entering and clogging cannula 190. Cannula obturator 192 also has a generally flat profile to match the generally flat profile of cannula 190. If used, cannula obturator 192 is removed from cannula 190 prior to inserting hook 170 into cannula 190 for insertion into the patient.

Cannula 190 also includes a rounded blunt tip 194. Although not shown, cannula 190 can include a female lure or similar type connection, distal from blunt tip 194 and obturator 192 can include a male lure connection to releasably couple operator 192 to needle 190 during insertion. Similar to cannula 180 and hook obturator 183, a hook obturator 193, shown in FIG. 12A, can be used with cannula 190. Hook obturator 193 has a hook 195 that can be manipulated in the manner described above with respect to hook 185.

Referring to FIG. 13, to perform a non-invasive facelift procedure according to an exemplary embodiment of the present invention, the surgeon makes an incision in the mouth 60 in the intraoral mucosa 62 above bone 50, shown by arrow “B”. The layers of soft tissue above intraoral mucosa 62, namely fat/deep fat pad 64 (where present), muscle 66, SMAS 68, fat 70, and skin 72, are lifted in the procedure.

After the incision is made in each oral mucosa 62, depending on where the lift is being performed, any one of plates 110, 130, 140, 140′, 140″ is inserted into the incision and secured to bone 50, such as by screws or tacks.

After the plate is secured into place, the surgeon then inserts the desired hook 150, 160, 170 percutaneously through the exterior of skin 72, through fat 70, SMAS 68, muscle 66, and fat/deep fat pad 64. Hook 150, 160, 170 is manipulated to a desired location and pull backward to lift the skin a desired amount. Hook 150, 160, 170 is then secured to the inserted plate 110 via sutures or retrieved if desired after anchoring tissue to the plate 110. In this method, the only penetration of the exterior of the skin is the needle insertion, thereby reducing or total eliminating scarring after the procedure.

Optionally, the surgeon utilizes CT scanning, cone beam CT scanning, or guided device to provide real-time visualization and details of all elements being inserted and/or implanted into the patient. The use of CT scanning allows the surgeon to have a real-time view of the procedure and to be able to see and avoid particular areas of the patient's anatomy. Alternatively, the surgeon can omit the CT scan and perform the procedure manually.

The inventive procedure is especially useful when performing lifts in delicate areas of the face, such as around the, infraorbital and/or the mental foramen in the upper and/or lower jaw. The mental infraorbital foramen serves as a nerve passage through the bone to the soft tissue exterior of the bone. While prior are lifts performed by making incisions through the exterior of the skin, thereby risking an accidental cut of the foramen nerve, the present invention, particularly using CT scan, markedly reduces the risks of making such an accidental cut or injury. There is no excision of tissue and the inventive procedure provides for repositioning the natural anatomy of the soft tissues to where it once belonged.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A system for performing minimally invasive facelifts, the system comprising:
a. a plate having:
i. a base having a top surface and an opposing bottom surface, the base having a plurality of through holes formed therein and extending between the top surface and the bottom surface; and
ii. a plurality of barbs extending outwardly from the bottom surface;
b. at least one anchor screw adapted to secure the plate to the bone of patients.

2. The system according to claim 1, wherein at least one of the plurality of barbs extends in a first direction at a first angle away from the bottom surface, and at least another of the plurality of barbs extends in a second direction and a second angle, different from the first angle.

3. The system according to claim 2, wherein the first angle is generally orthogonal relative to the bottom surface.

4. The system according to claim 1, wherein at least one of the top surface and a bottom surface extends in three dimensions.

5. The system according to claim 4, further comprising a filler material applied to the at least one of the top surface and the bottom surface.

6. The system according to claim 1, further comprising a hook for performing facelifts, the hook comprising:
a. a generally cylindrical body extending along a longitudinal axis, the body having:
i. a first end having a tip extending away from the body along the longitudinal axis;
ii. a second end having a through opening formed therein;
iii. a plurality of barbs extending from the body; and
b. a suture having a first end extending through the through opening and secured to the second end.

7. The system according to claim 6, wherein the plurality of barbs comprises a first set of barbs located proximal to the first end and extending toward the second end and a second set of barbs located proximal to the second end and extending toward the first end.

8. The system according to claim 6, further comprising a hook for performing facelifts, the hook comprising:
a. an elongate, generally planar body extending along a longitudinal axis, the body having:
i. a first face;
ii. a second face, opposing the first face, the body having a plurality of through openings extending between the first face and a second face;
b. a first plurality of barbs extending outwardly from the first face in a first direction generally along the longitudinal axis; and
c. a suture fixedly connected to an end of the body.

9. The system according to claim 8, wherein the hook further comprises a second plurality of barbs extending outward from the second face in a second direction, different from the first direction, generally along the longitudinal axis.

10. The system according to claim 8, further comprising an introducer cannula sized to allow the body to be inserted therein.

11. The system according to claim 10, further comprising an obturator sized to fit inside the introducer cannula, the obturator having a distal end, the distal end having a hook.

12. A method performing a non-invasive facelift procedure on a patient comprising the steps of:
a. making an incision inside the patient's mouth in intraoral mucosa, above bone;
b. inserting a plate into the incision and securing the plate to the bone;
c. inserting hook percutaneously through exterior of the patient's skin, through fat, SMA, muscle, and fat slap deep fat pad; and
d. manipulating the hook to a desired location, and, pulling the hook backward to lift the skin a desired amount.

13. The method according to claim 12, further comprising the step of securing the hook to the plate.

14. The method according to claim 12, further comprising the steps of:
e. anchoring the lifted skin to the plate; and
f. removing the hook from the patient.
